(19) United States
(12) Patent Application Publication
(10) Pub. No.: US 2005/0053551 A1
(43) Pub. Date: Mar. 10, 2005

(54) Use of a combination of gadolinium-based contrast media and iodinated contrast media as a contrast agent for X-ray based medical imaging procedures

(76) Inventor: Carlos M. Badiola, West Hartford, CT (US)

Correspondence Address: ST. ONGE STEWARD JOHNSTON & REENS, LLC, 986 BEDFORD STREET, STAMFORD, CT, 06905-5619, US

(21) Appl. No.: 10/936,608

(22) Filed: Sep. 8, 2004

Related U.S. Application Data
(60) Provisional application No. 60/501,115, filed on Sep. 8, 2003.

(51) Int. Cl. A61K 49/00

(52) U.S. Cl. 424/9.36; 424/9.37

(57) ABSTRACT

This invention relates to graphic visualization for X-ray based medical imaging procedures such as angiography, computed tomography, intravenous urography, cholangiography, and CT scans with use of contrast media. The contrast media is a mixture of a gadolinium contrast constituent and an iodinated contrast constituent.

### PRIOR APPLICATIONS

Applicant claims priority benefits under 35 U.S.C. § 119 (e) of U.S. Provisional Patent Application Ser. No. 60/501,115 filed Sep. 8, 2003 which is hereby incorporated by reference.

### FIELD OF THE INVENTION

This invention relates to the use of contrast media for X-ray based medical imaging procedures such as angiography, computed tomography, intravenous urography, cholangiography, and CT scans.

### BACKGROUND

Iodinated contrast media is routinely used as a contrast agent during the performance of medical imaging procedures involving X-rays. The most common applications include angiography, computed tomography, intravenous urography, cholangiography, and CT scans.

One of the major and potentially life-threatening side effects of the use of intravascular iodinated contrast media is contrast nephrotoxicity. This can be defined as a sudden change of renal status after the administration of iodinated contrast media when no other etiology appears likely from the clinical record. The effect can be transitory or permanent. If permanent, it may result in irreversible renal dysfunction and the subsequent need for dialysis. The exact pathogenesis of the iodinated contrast media induced nephrotoxic effect is unknown. There are several identifiable risk factors that may predispose patients to iodinated contrast induce nephrotoxicity. The most important appear to be diabetes and pre-existing renal insufficiency. Several strategies are available in order to minimize the risk of iodinated contrast nephrotoxicity. These include, the use of low osmolality iodinated contrast media, reducing the volume of administered iodinated contrast, hydration, the use of drugs that may reduce the risk of nephrotoxicity, as well as the use of alternative, non-nephrotoxic contrast media.

In response to the potential nephrotoxic effects of iodinated contrast media, alternative, non-nephrotoxic intravascular contrast agents have increasingly been utilized during the performance of medical imaging procedures that require the use of intravascular contrast agents. Both gadolinium-based contrast agents and carbon dioxide have been used as intravascular contrast agents in an effort to avoid the potential nephrotoxic effects of iodinated contrast. Gadolinium-based contrast agents are currently marketed for use during contrast-enhance magnetic resonance imaging (MRI) studies. Current published data suggests that gadolinium based contrast agents are non-nephrotoxic when injected intraarterialy or intravenously at doses up to 0.4 mmol/Kg of body weight. There is extensive published experience in the radiology literature supporting the use of gadolinium as an alternative contrast agent for both arteriography, including cerebral arteriography, and venography. There is also limited published data showing the potential value of gadolinium as an intravascular contrast agent for computed tomography.

One of the major limitations of the use of gadolinium as an alternative contrast agent is the inferior quality of the gadolinium-enhanced images when compared to iodinated contrast enhanced images. Gadolinium-enhanced images obtained during angiography and computed tomography are of inferior quality when compared to those achievable with iodinated contrast media. This is due to the low concentration of the gadolinium ion in commercially available preparations as well as the limited volume of gadolinium-based agents that can be safely administered into the circulation. The inferior diagnostic quality of the gadolinium-based imaging limits the usefulness of gadolinium-based contrast agents as alternative contrast media during the performance of angiography and computed tomography.

Kopka et al., MR arthrography of the shoulder with gadopentetate dimeglumine: influence of concentration, iodinated contrast material, and time on signal intensity. AJR 1994; 163: 621-623 (herein incorporated by reference) described the mixing of gadolinium-based contrast and iodinated contrast during MRI arthrography. During the performance of an MRI arthrogram, a needle first needs to be advanced into the joint space under fluoroscopic (X-ray) guidance in order to inject a dilute mixture of the gadolinium-based agent into the joint space. As the injected dilute gadolinium-based contrast agent is not visible on fluoroscopic images, the addition of small quantities of iodinated contrast, which can be easily visualized with fluoroscopy, is used in order to ascertain that the needle used for the injection was appropriately positioned within the joint prior to the gadolinium-based contrast injection. This practice raised the question of potentially deleterious and unknown interactions between the two agents. Of greatest concern was the potential for dissociation of the gadolinium ion from the gadolinium complex available for injection, as the free ion is systemically toxic. Despite this, the use of mixing of small quantities of iodinated contrast media with gadolinium-based contrast has become routine practice during the performance of magnetic resonance arthrography by radiologists in the United States. No apparent deleterious effects have been reported related to this practice.

Brown et al., Is a mixture of gadolinium and iodinated contrast material safe during MR arthrography? AJR 2000; 175: 1087-1090 (herein incorporated by reference) performed a spectroscopic study in order to determine whether free gadolinium ion can dissociate from a commercially available gadolinium-based contrast agent when mixed with iodinated contrast agent or other solutions routinely used during MRI arthrography. Brown reported no significant dissociation of the gadolinium ion when gadopentetate dimeglumide was mixed with several different commercially available iodinated contrast agents.

During the performance of arteriography with gadolinium, it may become necessary to perform separate injections of iodinated contrast. The additional use of the iodinated contrast media may be required due to inadequate vascular visualization with the use of a gadolinium-based agent. This addition of iodinated contrast, as separate intravascular injections, with subsequent mixing of the gadolinium-based agent and the iodinated contrast agent within the circulation, has been reported in the literature and is also likely widely used in clinical practice. See for example, Hammer FD et al. Galolinium dimeglumine: an alternative contrast agent for digital subtraction angiography. Eur. Radiol. 1999: 9 (1): 128-36 (herein incorporated by reference). No apparent deleterious effects from the mixing of the two agents in the intravascular space have been reported.

Accordingly, what is needed is improvement of poor image X-ray image quality in procedures such as angiography, computed tomography, intravenous urography, cholangiography, and CT scans. It has been found that gadolinium-based agents can be mixed with iodine-based contrast media. This mixture results in marked improvement in image quality over the gadolinium-based agent used alone, while at the same time minimizing the volume of administered iodinated contrast.

### SUMMARY OF THE INVENTION

It is the object of the present invention to improve X-Ray image quality over the gadolinium-based agent used alone, while at the same time minimizing the total volume of administered iodinated contrast.

It is the object of the present invention to increase the radiographic density of gadolinium contrast while minimizing the total amount of iodinated contrast.

It is the object of the present invention to improve image quality of gadolinium radiography.

It is the object of the present invention to provide a method of imaging mammals susceptible to nephrotoxicity caused by contrast media.

It is the object of the present invention to reduce the likelihood of a patient undergoing dialysis as a result of being administered an X-Ray contrast agent.

It is the object of the present invention to provide a method of using contrast media for X-ray based medical imaging procedures such as angiography, computed tomography, intravenous urography and cholangiography.

It is the object of the present invention to improve state-of-the art medical X-Ray imaging procedures.

These and other objects of the present invention are met by providing a mixture of gadolinium-based contrast media and iodinated contrast media as a contrast agent for X-ray based medical imaging procedures. Preferebly, the method comprises administering to a patient in need thereof a contrast agent comprising a gadolinium contrast constituent and an iodinated contrast constituent. Preferably, the method includes the step of administering the contrast agent by injecting the patient with between about 15 ml to about 100 ml of contrast agent. Most preferably, the step of administering the contrast agent further comprises injecting the patient with about 20 ml of contrast agent. Optionally, the step of administering the contrast agent further comprises injecting the patient at a rate of approximately 10 ml per second for a total of approximately two seconds. Most preferably, the contrast agent comprises approximately four parts gadolinium contrast constituent, and approximately one part iodinated constituent. Optionally, contrast agent may comprise approximately three parts gadolinium contrast constituent, and approximately one part iodinated constituent. Preferably, the contrast agent comprises a larger percentage by weight of the total weight of the contrast agent than the iodinated constituent. The method is useful for patients that are in need of X-ray medical imaging analysis, including those selected from the group consisting of angiography, computed tomography, intravenous urography, cholangiography, CT scan and combinations thereof.

Other objects of the present invention are obtained by providing a method of improving X-Ray image quality by administering to a patient in need thereof a contrast agent comprising a gadolinium contrast constituent and an iodinated contrast constituent. Optionally, the step of administering the contrast agent further comprises injecting the patient with between about 15 ml to about 100 ml of contrast agent. Most preferably, the step of administering the contrast agent further comprises injecting the patient with about 20 ml of contrast agent. Optionally, the step of administering the contrast agent further comprises injecting the patient at a rate of approximately 10 ml per second for a total of approximately two seconds. Preferably, the contrast agent comprises approximately four parts gadolinium contrast constituent, and approximately one part iodinated constituent. Optionally, the contrast agent may comprise approximately three parts gadolinium contrast constituent, and approximately one part iodinated constituent. Preferably, the contrast agent comprises a larger percentage by weight of the total weight of the contrast agent than the iodinated constituent. Preferably, the method is used on patients in need of an X-ray medical imaging analysis such as those selected from the group consisting of angiography, computed tomography, intravenous urography, cholangiography, CT scan and combinations thereof.

Other objects of the present invention are obtained by providing a method of medically imaging a mammal susceptible to nephrotoxicity comprising administering to the mammal a contrast agent comprising a gadolinium contrast constituent and an iodinated contrast constituent. Optionally, the step of administering the contrast agent further comprises injecting the mammal with between about 15 ml to about 100 ml of contrast agent. Preferably, the step of administering the contrast agent further comprises injecting the mammal with about 20 ml of contrast agent. The step of administering the contrast agent further comprises injecting the mammal at a rate of approximately 10 ml per second for a total of approximately two seconds. Preferably, contrast agent comprises approximately four parts gadolinium contrast constituent, and approximately one part iodinated constituent. Optionally, the contrast agent comprises approximately three parts gadolinium contrast constituent, and approximately one part iodinated constituent. Preferably, the contrast agent comprises a larger percentage by weight of the total weight of the contrast agent than the iodinated constituent. Such methods are suitable wherein the mammal needs an X-ray medical imaging analysis, including those selected from the group consisting of angiography, computed tomography, intravenous urography, cholangiography, CT scan and combinations thereof.

The objects of the present invention are further obtained by providing a method of generating a medical image comprising: injecting a mammal with a contrast agent comprising a gadolinium contrast constituent, and an iodinated constituent; applying X-Rays to the mammal while the contrast agent is within the mammal; and generating an image of the internal structures of the mammal.

The objects of the present invention are obtainable due to a contrast agent for medical imaging comprising approximately four parts gadolinium contrast constituent, and approximately one part iodinated constituent. In related aspects of the embodiments described herein, the invention provides a diagnostic device and method for monitoring soft tissues in patients that may or may not be afflicted with disease together with a reagents for such imaging.

As used herein the term “administering”, means bringing the cells of the subject in contact with a contrast media as described herein. In most cases, such “administering”, takes place either take by subcutaneous or intramuscular injection of the contrast media composition into the subject.

As used herein, the terms “solution suitable for injection in a human patient”, “in a form suitable for injection in a human patient” and “pharmaceutically acceptable”, may be used interchangeably and refer to composition comprising carriers, diluents and reagents, capable of administration to a human subject without the production of adverse physiological effects.

The terms “treating”, “treatment” and “therapy” as used herein refer to curative therapy, prophylactic therapy, and preventative therapy.

The term “dose” as used herein refers to the amount of contrast media administered to a patient. The amount varies with the subject, as described below, however, is preferably about 20 ml for the humans.

The term “part” or “parts” as used herein refers to the amount of constituent by total volume.

These and other objects and features of the invention will be more fully appreciated when the following detailed description of the invention is read in conjunction with the accompanying drawings.

### BRIEF DESCRIPTION OF THE DRAWINGS

FIGS. 1(a) (b) and (c) are arteriograms of the left thigh of an 82-year-old woman with left lower extremity arterial insufficiency.

FIG. 2(a)(b) and (c) are arteriograms of the left thigh of a 50-year-old man.

FIG. 3 shows four plastic collection vials radiographed at 68 kVp (A, top row) and 90 kVp (B, bottom row).

### DETAILED DESCRIPTION OF THE DRAWINGS

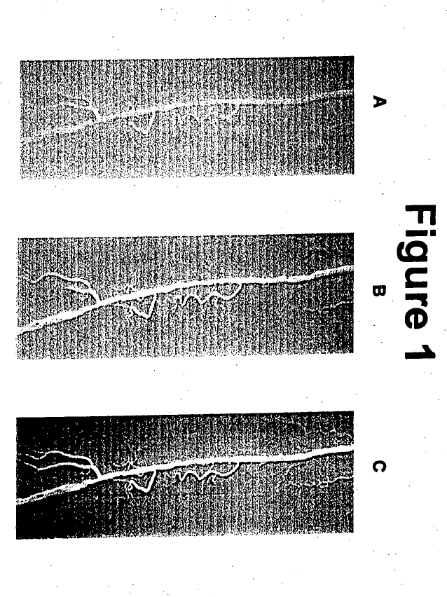

As described in the background gadolinium chelates are widely used as alternatives to iodinated contrast during DSA in patients considered to be at increased risk for contrast nephropathy. Because of relatively poor radiopacity, gadolinium chelates produce images of inferior quality relative to iodinated contrast. It has been found, and FIGS. 1, 2, and 3 demonstrate that the radiographic density and clinical image quality of gadolinium-based contrast is improved by adding a small amount of iodinated contrast to the gadolinium agent. A phantom study that assessed the relative radiographic densities of the gadolinium-iodinated contrast mixture, full strength gadolinium, full strength iodinated contrast and 20% iodinated contrast was conducted. Moreover, the clinical image quality of the mixture of gadolinium-based contrast and iodinated contrast was compared to full-strength gadolinium and full strength iodinated contrast during DSA.

FIGS. 1, 2, and 3 demonstrate the value of using a combination of gadolinium-based contrast media and iodinated contrast media as the contrast agent during X-ray based imaging procedures. Mixing small quantities of iodinated contrast with a gadolinium-based contrast agent results in marked improvement of angiographic image quality when compared to images obtained using the gadolinium-based agent alone. The invention yields diagnostic angiograms, with marked improvement in image quality when compared to images obtained with the gadolinium-based agent used alone.

The diagnostic quality of the images produced with the mixture of gadolinium-based contrast with iodinated contrast is comparable to the images produced with the use of undiluted iodinated contrast. The invention comprising gadolinium based contrast and iodinated contrast thus overcomes the major limitation of the gadolinium-based agent when used for angiography, poor image quality. The use of the inventive mixture also continues to offer a strategy that potentially reduces the risk of contrast induced nephrotoxicity by significantly reducing the administered volume of iodinated contrast media needed for the examination.

Preliminary clinical data, as well as in vitro data, suggests the invention provides a safe and useful combination of contrast media for intravascular use. This combination of contrast agents is applicable for use in patients at risk for contrast induced nephrotoxicity in whom it may be necessary to perform intravascular contrast enhanced X-ray imaging examinations, including, but not limited to, angiography and computed tomography.

The purpose of the producing the arteriograms shown in FIGS. 1, 2, and 3 was to determine if the image quality of gadolinium digital subtraction angiography (DSA) can be improved by the addition of small quantities of iodinated contrast to gadolinium.

The optical density (O.D.) of a mixture of four parts gadolinium-based contrast to one part iodinated contrast was measured through a phantom study and compared to that of full-strength gadolinium, full strength iodinated contrast, and a 20% solution of iodinated contrast. The clinical image quality of the mixture of gadolinium-based contrast and iodinated contrast was compared to the full-strength gadolinium and full strength iodinated contrast during DSA.

Overall, the DSA image quality of the gadolinium-iodinated contrast mixture was significantly improved relative to images obtained with full-strength gadolinium and compared favorably to that obtained with full strength iodinated contrast. The phantom data showed that the O.D. of the gadolinium-iodinated contrast mixture was much greater than that of full strength gadolinium and the 20% iodinated contrast solution. The increase in O.D. was greater than that expected from a simple additive effect of the O.D. of the contrast agents.

Ultimately it was determined that adding a small amount of iodinated contrast to gadolinium results in a significant improvement in the radiographic density and DSA image quality of gadolinium. This simple technique appears to overcome one of the major limitations of gadolinium-based angiography, poor radiographic density, while continuing to minimize the volume of administered iodinated contrast.

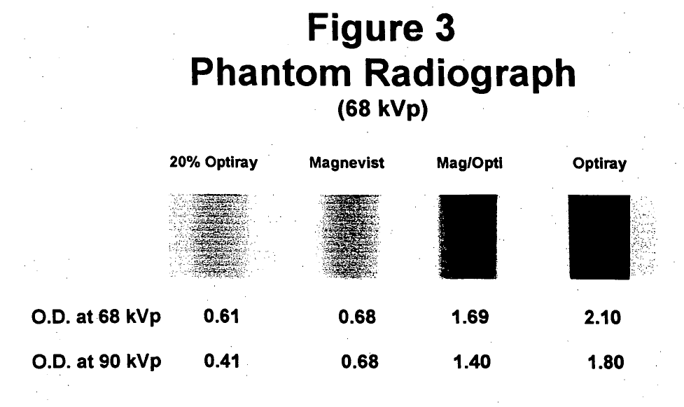

Referring now to FIG. 3, four phantoms were radiographed in a water bath using digital radiography at 68 and 90 kVp. Phantom # 1 contained a mixture of 1 part Optiray 320 (Mallinckrodt, St. Louis, Mo.) to 4 parts water, phantom #2 contained Magnevist (Berlex, Wayne, N.J.), phantom # 3 contained a mixture of 4 parts Magnevist to 1 part Optiray 320 and phantom # 4 Optiray 320. The optical density of the various contrast solutions was then measured with a densitometer (X-Rite 331, X-Rite Inc., Grandville, Mich.).

Still referring to FIG. 3, the mixture of four parts Magnevist to one part Optiray 320 was used for lower extremity DSA in three azotemic patients. In each patient, after suboptimal lower extremity arterial opacification with full-strength Magnevist, repeat DSA (Siemens Polystrar, 1024×1024 matrix, 2 frames/sec, automatic KVp settings) was performed using the mixture of four parts Magnevist to one part Optiray 320 followed by DSA with full-strength Optiray 320. The DSA images consisted of hand injections of 8 cc of the different contrast agents injected into the superficial femoral artery after contralateral catheterization with a 5 F catheter. In each patient, DSA imaging parameters were identical except for the use of the different contrast agents. Relative image quality was assessed by visual examination of the radiographic contrast of the different agents as well as the extent of peripheral branch visualization.

Still referring to FIG. 3, the O.D. of the 4 vials containing the mixture of 1 part Optiray 320 to 4 parts normal saline, Magnevist, the mixture of 4 parts Magnevist to 1 part Optiray 320 and finally Optiray 320 (vials 1-4 respectively) was performed. At 90 KVp, vials 1-4 showed optical densities of 0.41, 0.68, 1.40 and 1.80 respectively. At 68 KVp these values were 0.61, 0.68, 1.69 and 2.10.

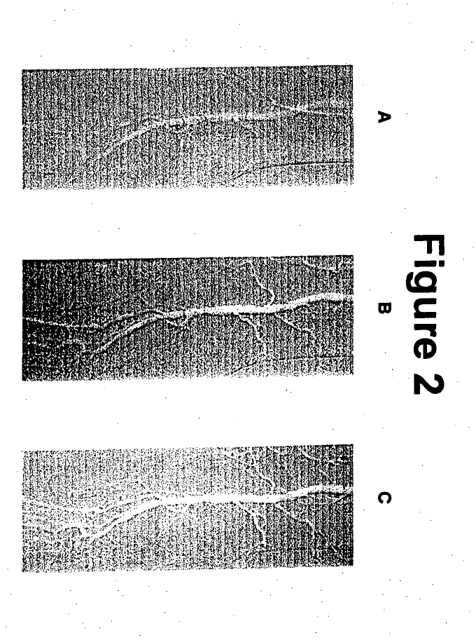

The mixture of Magnevist and Optiray 320 resulted in diagnostic angiographic images in all cases. The image quality obtained with the mixture of Magnevist and Optiray 320 was significantly improved relative to the image quality of full-strength gadolinium (FIGS. 1 and 2), and appeared marginally inferior to that observed with full strength Optiray 320 (FIGS. 1 and 2). Serum creatinine measurements in all three studied patients were stable at 24 and 48 hours. No complications were observed.

Gadolinium-based contrast agents are widely used as an alternative intraarterial contrast agent for DSA in patients at risk for contrast nephropathy. These contrast agents appear to be non-nephrotoxic at doses up to 0.4 mmol/Kg of body weight. The use of gadolinium chelates as alternative DSA contrast agents is limited by poor radiopacity. This is due to the low concentration of the gadolinium ion in commercially available preparations.

Issues regarding the safety of mixing gadolinium and iodinated contrast remain to be fully elucidated. In vitro spectroscopic analyses have shown no significant dissociation of the gadolinium ion when Magnevist was mixed with several different iodinated contrast agents. Currently, mixtures of gadolinium and iodinated contrast are widely used during shoulder MR arthrography, although at much lower volumes and gadolinium concentrations. Low doses of iodinated contrast are commonly used intravenously in patients with renal insufficiency to calculate GFR without observable detrimental effects on renal function.

The data shows that the mixing of small quantities of iodinated contrast with Magnevist results in a surprisingly significant increase in the radiographic contrast of Magnevist. The observed increase in O.D. is greater than that expected from a simple additive effect of the densities of the two contrast agents. The addition of small quantities of iodinated contrast to gadolinium may thus surprisingly aid in overcoming the major limitation of gadolinium radiography, poor radiopacity, while continuing to minimize the total dose of administered iodinated contrast. FIGS. 1, 2, and 3 demonstrate that this combination of contrast agents may be of clinical benefit for use in patients at risk for contrast induced nephropathy during conventional angiography and CT.

FIGS. 1(a) (b) and (c) are arteriograms of the left thigh of an 82-year-old woman with left lower extremity arterial insufficiency. Serum creatinine level was 2.9 mg/dl. The arteriograms of the left thigh were obtained after hand injection of a total of 8 cc of Magnevist (FIG. 1(a)), a combination of 4 parts Magnevist to 1 part Optiray 320 FIG. 1(b), and full strength Optiray 320 FIG. 1(c). Note improvement in contrast of the Magnevist-Optiray combination FIG. 1(b) when compared to Magnevist FIG. 1(a).

FIGS. 2(a)(b) and (c) are arteriograms of the left thigh of a 50-year-old man. Serum creatinine was 2.3 mg/dl. Arteriograms of the left lower extremity obtained after hand injection of a total of 8 cc of Magnevist FIG. 2(a), a combination of 4 parts Magnevist to 1 part Optiray 320 FIG. 2(b) and full strength Optiray 320 FIG. 2(c). As in FIG. 1, note improvement in contrast of the Magnevist-Optiray 320 combination FIG. 2(b) when compared to Magnevist FIG. 2(a).

FIG. 3 shows a eries of four plastic collection vials radiographed at 68 kVp (A, top row) and 90 kVp (B, bottom row). These are numbered 14 from left to right and contain:
1. One part Optiray 320 and 4 parts water.
2. Magnevist
3. One part Optiray 320 and 4 parts Magnevist.
4. Optiray 320.

Note the marked increase in radiographic density of the vial containing the combination of Magnevist and Optiray (number 3) when compared to Magnevist (number 2) at both kVp settings. Note also the increased density of vials numbers 1 and 3 (those containing iodinated contrast) at the lower kVp setting.

In the preferred embodiment of the present invention, the contrast agent is used comprising a gadolinium contrast constituent and an iodinated contrast constituent. These two constituents may be supplied from any known supplier of gadolinium contrast agents, and iodinated contrast agents. Preferably, both constituents are pharmaceutically acceptable. As described above, it is the mixture of both constituents that is believed to result in the increase of radiographic density of the gadolinium constituent, while minimizing the total amount of iodinated contrast. This objective is believed to be met by providing a mixture of between about 10 parts by volume of gadolinium contrast constituent, mixed with 1 part by volume of iodinated contrast constituent (about 10:1 ratio). It is also believed that the objectives of the present invention are also met by providing a mixture of between about 2.5 parts by volume of gadolinium contrast constituent, mixed with 1 part by volume of iodinated contrast constituent (about 2.5:1 ratio). It is also believed that the objectives of the present invention are also met by providing a mixture of between about 7.5 parts by volume of gadolinium contrast constituent, mixed with 3 part by volume of iodinated contrast constituent (about 7.5:3 ratio). Preferably, as supported by the data, it is believed that the objectives of the present invention are also met by providing a mixture of between about 4 parts by volume of gadolinium contrast constituent, mixed with 1 part by volume of iodinated contrast constituent (about 4:1 ratio).

Obviously, many modifications may be made without departing from the basic spirit of the present invention. Accordingly, it will be appreciated by those skilled in the art that within the scope of the appended claims, the invention may be practiced other than has been specifically described herein.

1. A method of using a contrast agent comprising administering to a patient in need thereof a contrast agent comprising a gadolinium contrast constituent and an iodinated contrast constituent.

2. The method of claim 1 wherein the step of administering the contrast agent further comprises injecting the patient with between about 15 ml to about 100 ml of contrast agent.

3. The method of claim 1 wherein the step of administering the contrast agent further comprises injecting the patient with about 20 ml of contrast agent.

4. The method of claim 1 wherein the step of administering the contrast agent further comprises injecting the patient at a rate of approximately 10 ml per second for a total of approximately two seconds.

5. The method of claim 1 wherein the contrast agent comprises approximately 10 parts gadolinium contrast constituent, and approximately 1 part iodinated constituent.

6. The method of claim 1 wherein the contrast agent comprises approximately 7.5 parts gadolinium contrast constituent, and approximately 3 part iodinated constituent.

7. The method of claim 1 wherein the contrast agent comprises approximately 4 parts gadolinium contrast constituent, and approximately 1 part iodinated constituent.

8. The method of claim 1 wherein the contrast agent comprises approximately 2.5 parts gadolinium contrast constituent, and approximately 1 part iodinated constituent.

9. The method of claim 1 wherein the contrast agent comprises approximately three parts gadolinium contrast constituent, and approximately one part iodinated constituent.

10. The method of claim 1 wherein the contrast agent comprises a larger percentage by weight of the total weight of the contrast agent than the iodinated constituent.

11. The method of claim 1 wherein the patient needs an X-ray medical imaging analysis.

12. The method of claim 11 where the X-ray medical imaging analysis is selected from the group consisting of angiography, computed tomography, intravenous urography, cholangiography, CT scan and combinations thereof.

13. A method of improving X-Ray image quality administering to a patient in need thereof a contrast agent comprising a gadolinium contrast constituent and an iodinated contrast constituent.

14. The method of claim 13 wherein the step of administering the contrast agent further comprises injecting the patient with between about 15 ml to about 100 ml of contrast agent.

15. The method of claim 13 wherein the step of administering the contrast agent further comprises injecting the patient with about 20 ml of contrast agent.

16. The method of claim 13 wherein the step of administering the contrast agent further comprises injecting the patient at a rate of approximately 10 ml per second for a total of approximately two seconds.

17. The method of claim 13 wherein the contrast agent comprises approximately four parts gadolinium contrast constituent, and approximately one part iodinated constituent.

18. The method of claim 13 wherein the contrast agent comprises approximately three parts gadolinium contrast constituent, and approximately one part iodinated constituent.

19. The method of claim 13 wherein the contrast agent comprises a larger percentage by weight of the total weight of the contrast agent than the iodinated constituent.

20. The method of claim 13 wherein the patient needs an X-ray medical imaging analysis is selected from the group consisting of angiography, computed tomography, intravenous urography, cholangiography, CT scan and combinations thereof.

21. A method of medically imaging a mammal susceptible to nephrotoxicity comprising administering to the mammal a contrast agent comprising a gadolinium contrast constituent and an iodinated contrast constituent.

22. The method of claim 21 wherein the step of administering the contrast agent further comprises injecting the mammal with between about 15 ml to about 100 ml of contrast agent.

23. The method of claim 21 wherein the step of administering the contrast agent further comprises injecting the mammal with about 20 ml of contrast agent.

24. The method of claim 21 wherein the step of administering the contrast agent further comprises injecting the mammal at a rate of approximately 10 ml per second for a total of approximately two seconds.

25. The method of claim 21 wherein the contrast agent comprises approximately four parts gadolinium contrast constituent, and approximately one part iodinated constituent.

26. The method of claim 21 wherein the contrast agent comprises approximately three parts gadolinium contrast constituent, and approximately one part iodinated constituent.

27. The method of claim 21 wherein the contrast agent comprises a larger percentage by weight of the total weight of the contrast agent than the iodinated constituent.

28. The method of claim 21 wherein the mammal needs an X-ray medical imaging analysis.

29. The method of claim 28 where the X-ray medical imaging analysis is selected from the group consisting of angiography, computed tomography, intravenous urography, cholangiography, CT scan and combinations thereof.

30. A method of generating a medical image comprising:
injecting a mammal with a contrast agent comprising a gadolinium contrast constituent, and an iodinated constituent;
applying X-Rays to the mammal while the contrast agent is within the mammal;
generating an image of the internal structures of the mammal.

31. A contrast agent for medical imaging comprising a gadolinium contrast constituent, and an iodinated constituent wherein the gadolinium contrast constituent is mixed with the iodinated constituent in a ratio between the range of about 10:1 parts to about 2.5 to 1 parts, wherein the density of the gadolinium contrast in increased.
